(19) United States
(12) Patent Application Publication
(10) Pub. No.: US 2026/0257008 A1
(43) Pub. Date: Sep. 3, 2026

(54) On-body injection device

(71) Applicant: SHL Medical AG, Zug (CH)

(72) Inventors: Lukas Bannwart, Zug (CH); René Zander, Zug (CH); Torbjörn Neby, Nacka Strand (SE)

(21) Appl. No.: 18/871,309

(22) PCT Filed: May 19, 2023
(86) PCT No.: PCT/EP2023/063460

§ 371 Date: Dec. 3, 2024

Related U.S. Application Data
(60) Provisional application No. 63/348,496, filed on Jun. 3, 2022.

(30) Foreign Application Priority Data
Jul. 19, 2022 (EP) 22185848.3

(51) Int. Cl. A61M 5/142; A61M 5/145; A61M 5/165; A61M 5/31; A61M 5/32

(52) U.S. Cl. CPC A61M 5/14248; A61M 5/14526; A61M 5/165; A61M 5/3287; A61M 2005/14252; A61M 2005/1652; A61M 2005/3123

(57) ABSTRACT

The present disclosure provides an on-body injection device including a housing provided with a medicament inlet, a movable member channel arranged in the housing, and a movable member arranged in the movable member channel. The movable member channel includes a fluid inlet in fluid communication with the medicament inlet, and a fluid outlet. The on-body injection device further includes a skin penetration device configured to move from a starting position to a skin penetration position, a conduit connected to the skin penetration device and to the fluid outlet, and a movable structure pretensioned in a direction away from the movable member.

### CROSS-REFERENCE TO RELATED APPLICATIONS

The present application is a U.S. National Phase Application pursuant to 35 U.S.C. §371 of International Application No. PCT/EP2023/063460 filed May 19, 2023, which claims priority to (i) U.S. Provisional Application No. 63/348,496 filed Jun. 3, 2022, and (ii) EP Patent Application No. 22185848.3 filed Jul. 19, 2022. The entire disclosure contents of these applications are herewith incorporated by reference into the present application.

### TECHNICAL FIELD

The present disclosure generally relates to medical devices, in particular to on-body injection devices.

### BACKGROUND

Some injection devices used today for various medicament administrations are worn by the user. If medicament administration with such a device is desired over a longer period of time, the injection device usually comprises a pump and a motor that drives the pump.

U.S. Pat. No. 11,185,629 B2 discloses a dosing apparatus for on-body use. The dosing apparatus comprises container, a rotary drive, a pump, a puncture cannula, and an indwelling cannula. The rotary drive drives a barrel cam, which initially blocks a pretensioned control element from moving. When the barrel cam is rotated, the control element is released. Movement of the control element causes a first and a second runner to move downwards. The puncture cannula runs in the longitudinal direction within the indwelling cannula. Movement of the first and the second runner cause the puncture cannula and the indwelling cannula to move concurrently with a respective one of the first runner and the second runner. As the control element is moved further, it causes the first runner to move back up. This results in the puncture cannula being withdrawn upward from the indwelling cannula.

Devices that comprise the drug to be administered, a motor, and pump, such as the one disclosed in U.S. Pat. No. 11,185,629 may be relatively large and may therefore be quite visible.

### SUMMARY

A general object of the present disclosure is to provide an on-body injection device that solves or at least mitigates the problems of the prior art.

There is hence according to a first aspect of the present disclosure provided an on-body injection device comprising: a housing provided with a medicament inlet, a movable member channel arranged in the housing, a movable member arranged in the movable member channel, the movable member channel having a fluid inlet in fluid communication with the medicament inlet, and a fluid outlet, wherein the movable member is configured to move in the movable member channel, by a built-up overpressure inside the movable member channel relative to a pressure on the movable member outside the movable member channel, from a first movable member position in which the movable member blocks the fluid outlet to a second movable member position in which the fluid outlet is in fluid connection with the fluid inlet, a skin penetration device configured to move from a starting position to a skin penetration position, a conduit connected to the skin penetration device and to the fluid outlet, and a movable structure pretensioned in a direction away from the movable member, wherein the movable member is configured to engage with the structure when the movable member is in the first movable member position, preventing the structure from moving away from the movable member, and wherein the movable member is configured to disengage from the structure when the movable member is moved towards the second movable member position, causing the pretensioned structure to move away from the movable member to an intermediate position, bringing the skin penetration device from the starting position to the skin penetration position.

The skin penetration device may thus be actuated by means of the pressure provided by the medicament that flows through the medicament inlet. The actuation may thus be completely passive. In particular, no motor is required in the on-body injection device for moving the skin penetration device. As a result, the on-body injection device may be provided as a standalone injection device that by means of a hose can be coupled to a medicament pump device containing a medicament container, a motor, and a pump driven by the motor configured to pump the medicament to the on-body injection device through the hose. The on-body injection device may therefore be made more compact, and it can be placed on the user more discretely.

The movable member may be configured to move from the first movable member position to the second movable member position by linear movement, by rotation, or by a combination of linear movement and rotation.

The movable member may be a piston.

The movable member channel may be a piston channel.

The structure may be a cam element.

In the present disclosure, when the term “distal direction” is used, this refers to the direction pointing away from the dose delivery site during use of the on-body injection device/medicament delivery device. When the term “distal part/end” is used, this refers to the part/end of the delivery device, or the parts/ends of the members thereof, which under use of the medicament delivery device is/are located furthest away from the dose delivery site.

Correspondingly, when the term “proximal direction” is used, this refers to the direction pointing towards the dose delivery site during use of the medicament delivery device. The body side of the device is the side of the device adjacent to the dose delivery site during use of the medicament delivery device.

When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component.

Similarly, “radial” or “radially” refer to a direction extending radially relative to the axis, and “rotation”, “rotational” and “rotationally” refer to rotation relative to the axis.

One embodiment comprises a gas filter configured to allow air to pass through and to block liquid from passing through, wherein the movable member has an internal channel extending from a lower end of the movable member along a longitudinal axis of the movable member, and wherein the internal channel is in fluid communication with the fluid inlet, and wherein the gas filter seals the internal channel.

According to one embodiment the structure is configured to move away from the movable member in a direction that is perpendicular to the movement of the movable member from the first movable member position to the second movable member position.

One embodiment comprises a sealing ring arranged around the movable member, the sealing ring being configured to provide fluid sealing between the movable member channel and the movable member.

According to one embodiment one of the movable member and the structure comprises a recess and the other one of the movable member and the structure has a heel configured to engage with the recess to engage the movable member and the structure in the first movable member position of the movable member.

According to one embodiment the housing has a body side wall configured to bear against the skin of a user, and a distal side wall opposite to the body side wall, wherein the movable member is configured to move in a direction from the body side wall towards the distal side wall when the movable member is moved from the first movable member position to the second movable member position.

According to one embodiment the body side wall is provided with a through-opening, wherein the skin penetration device comprises a cannula configured to move out from the housing via the through-opening when the skin penetration device is moved from the starting position to the skin penetration position.

According to one embodiment the housing has a sidewall connecting the body side wall and the distal side wall, wherein the structure is configured to move towards the sidewall.

According to one embodiment the structure has a first cam surface configured to cooperate with the skin penetration device to cause the skin penetration device to move from the starting position to the skin penetration position as the structure is moved to the intermediate position.

According to one embodiment the structure is by pretension configured to continue to move from the intermediate position to a final structure position, causing the structure to bring part of the skin penetration device from the skin penetration position to a final position.

According to one embodiment the final position is the same as the starting position.

According to one embodiment the structure has a second cam surface configured to cooperate with the skin penetration device to cause the skin penetration device to move from the skin penetration position to the final position.

There is according to a second aspect of the present disclosure provided a medicament delivery device comprising: the on-body injection device of the first aspect, a hose connected to the medicament inlet of the on-body injection device, and a medicament pump device comprising: a medicament pump device housing, a motor and a pump arranged in the medicament pump device housing, wherein the motor is configured to drive the pump, and wherein the pump is configured to pump medicament to the on-body injection device via the hose.

Generally, all terms used in the claims are to be interpreted according to their ordinary meaning in the technical field, unless explicitly defined otherwise herein. All references to “a/an/the element, apparatus, component, means, etc.” are to be interpreted openly as referring to at least one instance of the element, apparatus, component, means, etc., unless explicitly stated otherwise.

### BRIEF DESCRIPTION OF THE DRAWINGS

The specific embodiments of the inventive concept will now be described, by way of example, with reference to the accompanying drawings, in which:

FIG. 1 schematically shows an example of a medicament delivery device comprising an on-body injection device and a medicament pump device;

FIG. 2 schematically shows a cross-section of the on-body injection device in FIG. 1 through lines A-A;

FIG. 3 schematically shows a top view of the on-body injection device with a top part of its housing removed to expose the internal components; and

FIGS. 4-9 schematically show a side view of the medicament delivery device in FIG. 1, with a cross-section of the on-body injection device during various stages of medicament delivery.

### DETAILED DESCRIPTION

The inventive concept will now be described more fully hereinafter with reference to the accompanying drawings, in which exemplifying embodiments are shown. The inventive concept may, however, be embodied in many different forms and should not be construed as limited to the embodiments set forth herein; rather, these embodiments are provided by way of example so that this disclosure will be thorough and complete, and will fully convey the scope of the inventive concept to those skilled in the art. Like numbers refer to like elements throughout the description.

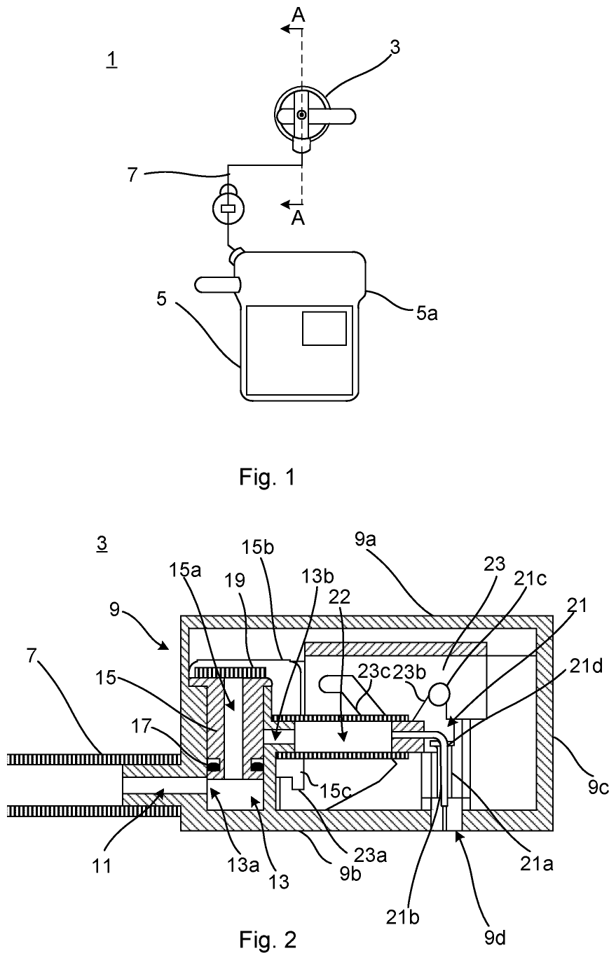

FIG. 1 depicts an example of a medicament delivery device 1.

The exemplified medicament delivery device 1 comprises an on-body injection device 3 and a medicament pump device 5.

The medicament delivery device 1 comprises a hose 7 which connects the on-body injection device 3 with the medicament pump device 5. The on-body injection device 3 and the medicament pump device 5 are thus two physically separate devices, which can be connected by the hose 7. Alternatively, the body injection device and the medicament pump device could be within a single housing.

The on-body injection device 3 is configured to be attached to the skin of a user. The on-body injection device 3 may for example be provided with an adhesive which makes the on-body injection device 3 adhere to a user's skin.

The on-body injection device 3 comprises one or more cannulas that can penetrate the skin and through which medicament may be administered.

The medicament pump device 5 may be an on-body device. Thus, the medicament pump device 5 may be configured to be attached to the skin of a user, for example by means of an adhesive.

The on-body injection device 3 and the medicament pump device 5 can be attached to different areas of a user's skin, with the length of the hose 7 determining the maximum distance between the on-body injection device 3 and the medicament pump device 5.

The medicament pump device 5 comprises a medicament container comprising a medicament. The medicament container may for example be in the form of a bag, a syringe, or a cartridge.

The medicament pump device 5 comprises a housing 5a. The medicament pump device 5 comprises an electric motor, an energy storage device such as a battery configured to power the electric motor, and pump configured to be driven by the electric motor, arranged in the housing 5a. The pump is configured to pump medicament from the medicament container to the on-body injection device 3 via the hose 7.

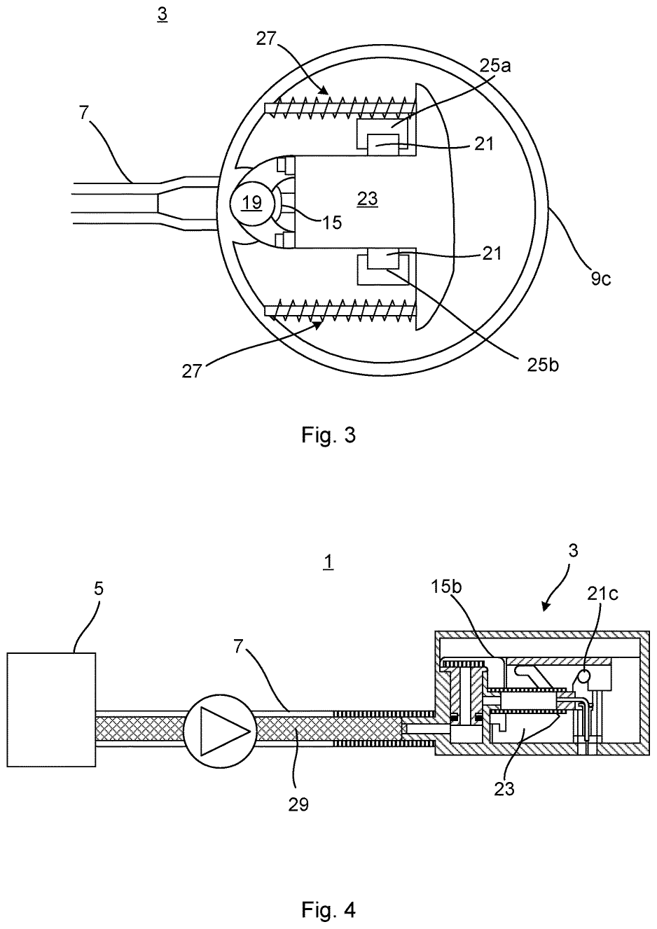

FIG. 2 shows a cross-section of the on-body injection device 3, along lines A-A in FIG. 1.

The on-body injection device 3 comprises a housing 9. The housing 9 includes a distal side wall 9a and a body side wall 9b. In use, the body side wall 9b faces the injection site. An outer surface of the body side wall 9b may be provided with an adhesive which allows the on-body injection device 3 to adhere to a user's skin. In use, the distal side wall 9a faces away from the injection site.

The body side wall 9b comprises a through-opening 9d through which a puncture cannula and an indwelling cannula can pass.

The housing 9 is provided with a medicament inlet 11. The medicament inlet 11 is configured to be connected to the hose 7. The medicament inlet 11 extends through a wall of the housing 9 and provides a fluid connection between the exterior and the interior of the housing 9.

The on-body injection device 3 comprises a movable member channel 13 arranged inside the housing 9. The movable member channel may be an integral part of the housing or may be a separate component attached directly or indirectly to the housing. According to the example, the movable member channel 13 is a piston channel. The movable member channel 13 has a proximal surface defined by the body side wall 9b of the housing 9. The movable member channel 13 extends from the body side wall 9b towards the distal side wall 9a of the housing 9.

The movable member channel 13 has a fluid inlet 13a, which is in fluid communication with the medicament inlet 11.

The movable member channel 13 has a fluid outlet 13b. The fluid outlet 13b is arranged axially offset from the fluid inlet 13a along a longitudinal axis of the movable member channel 13. The fluid outlet 13b is further away from the body side wall 9b of the housing 9 than the fluid inlet 13a.

Alternatively, the fluid inlet and the fluid outlet may be offset in the circumferential direction of the movable member channel, or they may be both circumferentially and axially offset relative to each other.

The fluid outlet 13b is according to the example provided on an inner surface of the movable member channel 13 which faces the fluid inlet 13a.

The on-body injection device 3 comprises a movable member 15 arranged in the movable member channel 13. According to the example, the movable member 15 is a piston. Alternatively, the movable member could be another shape, such as a ball, a plunger, or a disc. The movable member could be compressible (soft) or incompressible (rigid). The movable member could also be a rotatable component such as a flexible arm or a trapdoor. The movable member could comprise a membrane that gas can pass through but that liquid cannot pass through.

The movable member 15 provides sealing between the inner surface of the movable member channel 13 and the outer surface of the movable member 15. The on-body injection device 3 may comprise a sealing ring 17 arranged around the movable member 15, for example in a circumferential groove provided in the outer surface of the movable member 15.

The movable member 15 is configured to move in the movable member channel 13 from a first movable member position, shown in FIG. 2, to a second movable member position, shown in FIG. 9. The movable member 15 is configured to move in a direction from the body side wall 9b towards the distal side wall 9a when the movable member 15 is moved from the first movable member position to the second movable member position. The movable member 15 is thus linearly movable inside the movable member channel 13.

The moveable member channel is shown extending perpendicular to the dose delivery site, but could be orientated in a different direction. Additionally, as an alternative to linear movement of the moveable member, the movable member could be arranged to rotate, or to rotate and move linearly, in the movable member channel.

In the first movable member position, the movable member 15 blocks the fluid outlet 13b. Thus, in the first movable member position, the movable member 15 blocks the fluid inlet 13a from fluid communication with the fluid outlet 13b.

In the second movable member position, the movable member 15 is arranged such that the fluid inlet 13a is in fluid communication with the fluid outlet 13b. For example, a lower end of the movable member 15 may in this case have at least partly passed the fluid outlet 13b. The movable member 15 has an internal channel 15a which extends along the longitudinal axis of the movable member 15, from the lower end of the movable member 15 to an upper end of the movable member 15. The internal channel 15a forms a through-opening in the movable member 15.

The on-body injection device 3 comprises a gas filter 19. The gas filter 19 is fixed to the movable member 15, although this is optional, with the gas filter alternatively attached to another part of the on-body injection device-as the idea is that the gas filter should allow gas out of the fluid path (i.e. the path the fluid takes between a medicament container and a medicament delivery member (skin penetration device) such as a needle or a cannula) without allowing fluid out of the fluid path, the gas filter could also be elsewhere in the fluid path. The gas filter 19 is arranged to seal an end of the internal channel 15a distal from the body side of the on-body injection device. The gas filter 19 is configured to allow air to pass through it and to block liquid from passing through. The gas filter 19 may for example comprise a polytetrafluoroethylene (PTFE) membrane.

Alternatively, the gas filter could be arranged to seal the internal channel elsewhere on the internal channel rather than at an end of the internal channel 15a distal from the body side of the on-body injection device, such as at an end of the internal channel 15a proximal to the body side of the on-body injection device and/or anywhere between the axial ends of the internal channel. The gas filter could also be elsewhere along the fluid path. Particularly in cases where the movable member is not a piston (as mentioned above), it may be beneficial (or necessary) to move the gas filter 19 to elsewhere along the fluid path (or to provide a second gas filter elsewhere along the fluid path).

The on-body injection device 3 comprises a skin penetration device 21. The skin penetration device 21 may be of the type disclosed in U.S. Pat. No. 11,185,629, which in U.S. Pat. No. 11,185,629 comprises a first runner, a second runner movable relative to the first runner, a puncture cannula and an indwelling cannula as described in column 8, lines 4-60, which is incorporated herein by this reference.

The skin penetration device 21 comprises an indwelling cannula 21a and a puncture cannula 21b which is movably arranged within the indwelling cannula 21a.

The on-body injection device 3 comprises a conduit 22 which connects the fluid outlet 13b with the skin penetration device 21. In particular, the conduit 22 connects the fluid outlet 13b with the indwelling cannula 21a. The particular shape of the conduit as depicted is optional, and the conduit may be other sizes or shapes-at a minimum, the conduit is simply a fluid connection between the fluid outlet and the medicament delivery member (in this example a cannula).

The on-body injection device 3 comprises a movable structure 23. The structure 23 is in the present example a cam element. The structure 23 is configured to engage with the movable member 15 when the movable member is in the first movable member position. The structure 23 is thereby maintained fixed relative to the movable member 15 while the movable member 15 is in the first movable member position.

The movable member 15 has a structure 15b which is arranged outside the movable member channel 13, extending towards the body side wall 9b parallel with the movable member channel 13. The structure 15b is configured to move concurrently with the movable member 15 when the movable member 15 is moved from the first movable member position to the second movable member position.

According to the example, the structure 23 comprises a recess 23a and the movable member comprises a heel 15c configured to engage with the recess 23a. The heel 15c is arranged on the structure 15b. Alternatively, the movable member 15 could comprise the recess and the structure 23 could comprise the heel. Other means for engagement are also envisaged, as would be apparent to the skilled person.

The structure 23 is pretensioned in a direction away from the movable member 15. The structure 23 is configured to move away from the movable member 15 in a direction that is perpendicular to the movement of the movable member 15 from the first movable member position to the second movable member position. This direction is parallel with the body side wall 9b.

When the movable member 15 is moved from the first movable member position to the second movable member position, the movable member 15 disengages from the structure 23. This results in that the structure 23 moves away from the movable member 15. The structure 23 will in this case according to the example move towards one of the sidewalls 9c of the housing 9.

The skin penetration device 21 is arranged to move from a starting position to a skin penetration position. The structure 23 is configured to actuate the skin penetration device 21 from the starting position to the skin penetration position and from the skin penetration position to a final position.

The structure 23 has a first cam surface 23b configured to cooperate with the skin penetration device 21 to cause the skin penetration device 21 to move from the starting position to the skin penetration position as the structure 23 is moved to an intermediate position. The skin penetration device 21 has a first runner 21c attached to the puncture cannula 21b and a second runner 21d attached to the indwelling cannula 21a, configured to cooperate with the first cam surface 23b when the structure 23 is moved away from the movable member 15.

The structure 23 has a second cam surface 23c configured to cooperate with the skin penetration device 21 to cause the skin penetration device 21 to move from the skin penetration position to the final position when the structure 23 is moved from the intermediate position further towards the sidewall 9c.

The first cam surface 23b and the second cam surface 23c define planes that intersect each other.

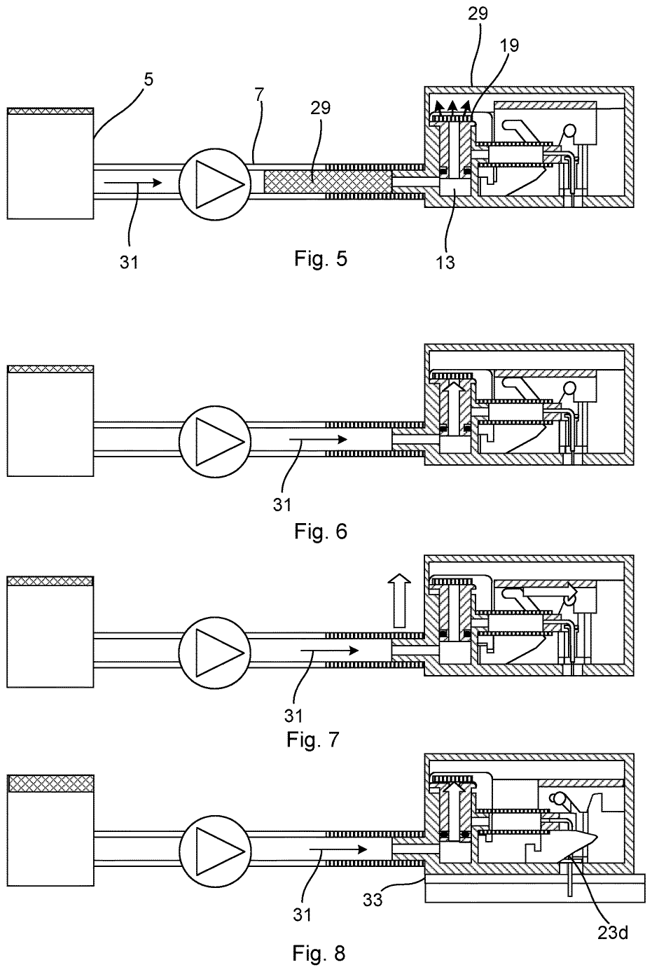

FIG. 3 shows a top view of the on-body injection device 3, with the distal side wall 9a of the housing 9 removed to expose internal components of the on-body injection device 3. The on-body injection device 3 comprises a structure with tracks 25a and 25b extending between the body side wall 9b and the distal side wall 9a. The first and second runners of the skin penetration device 21 are configured to run in the tracks 25a, 25b as they are moved from the starting position to the skin penetration position, and as the first runner is moved from the skin penetration position to the final position.

Further, the on-body injection device 3 comprises resilient members 27 which provide the pretension on the structure 23. The resilient members 27 may for example be springs such as coil springs. Alternatively, the on-body injection device 3 may comprise only one resilient member, or more than two resilient members.

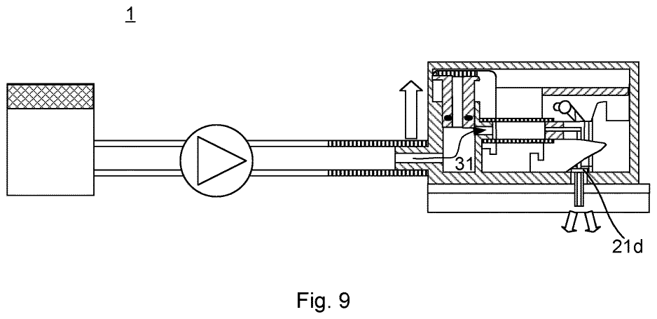

The operation of the medicament delivery device 1 will now be described with reference to FIGS. 4-9.

In FIG. 4, medicament delivery is about to commence. In this situation, the hose 7 may be filled with air 29.

The structure 23 is engaged with the movable member 15, which is in the first movable member position. The first runner 21c is interlocked with the structure 23. The skin penetration device 21 is in the starting position.

In FIG. 5, the pump of the medicament pump device 5 has started pumping the medicament 31 through the hose 7 to the on-body injection device 3. This pushes the air 29 through the medicament inlet 11 into the on-body injection device 3. The air 29 flows into the movable member channel 13, up through the internal channel 15a of the movable member 15, exiting the movable member 15 through the gas filter 19 as illustrated by the arrows.

In FIG. 6, the medicament 31 has entered into the movable member channel 13 through the medicament inlet 11 and flows up through the internal channel 15a towards the gas filter 19. Since the gas filter 19 does not let liquid pass through it, the movable member 15 is lifted inside the movable member channel 13 as shown in FIG. 7. The movable member 15 is thus moved from the first movable member position towards the second movable member position. Specifically, an overpressure is built up inside the movable member channel 13 relative to the pressure outside the movable member channel 13 due to the medicament 31 which accumulates inside the movable member channel 13, which causes the movable member 15 to move.

In FIG. 8, the movable member 15 has moved to such an extent towards the second movable member position such that it disengages from the structure 23. This results in that the structure 23 is moved in a direction away from the movable member 15. First, the first cam surface 23b cooperates with the first runner 21c and the second runner 21d, causing the skin penetration device 21 to move from the start position to the skin penetration position. In particular, both the first and second runners 21c and 21d are moved to the skin penetration position in a direction towards the body side wall 9b. This causes the puncture cannula 21b to puncture the injection site 33 and the indwelling cannula 21a to enter the injection site 33.

FIG. 8 illustrates when the structure 23 has moved from the intermediate position to a final structure position. As the structure 23 is moved from the intermediate position to the final structure position, the second cam surface cooperates with the first runner 21c, which is moved back in a direction towards the distal end wall 9a. As a result, the puncture cannula 21b is moved back into the housing 9. The indwelling cannula 21a is held in a position in which it penetrates the injection site 33, because a lower side 23d of the structure 23 holds the second runner 21d in the skin penetration position.

In FIG. 8, the movable member 15 has still not attained its second movable member position. In particular, the movable member 15 blocks the fluid outlet. Thus, the medicament 31 is not able to pass through the conduit 22 to the skin penetration device 21.

FIG. 9 shows when the movable member 15 has reached its second movable member position and fluid inlet 13a has been set in fluid communication with the fluid outlet 13b. The medicament 31 is thus able to pass through the fluid outlet 13b to the conduit 22 and the indwelling cannula 21a.

Optionally, the on-body device comprises a sensor that detects that the on-body injection device has been attached. Optionally, the sensor is a proximity sensor, a temperature sensor, a light level sensor, or a mechanical sensor such as a button. Optionally, the on-body device only allows injection to occur when it is detected that the on-body device has been attached (either with a physical limitation or with a digital limitation within a control system), or the on-body device initiates injection when it is detected that the on-body device has been attached.

The on-body injection devices/medicament delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary types of drugs that could be included in the medicament delivery devices described herein include, but are not limited to, antibodies, proteins, fusion proteins, peptibodies, polypeptides, pegylated proteins, protein fragments, protein analogues, protein variants, protein precursors, and/or protein derivatives. Exemplary drugs that could be included in the medicament delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-la (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatitis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)). Pharmaceutical formulations including, but not limited to, any drug described herein are also contemplated for use in the medicament delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

The inventive concept has mainly been described above with reference to a few examples. However, as is readily appreciated by a person skilled in the art, other embodiments than the ones disclosed above are equally possible within the scope of the inventive concept, as defined by the appended claims.

1-14. (canceled)

15. An on-body injection device comprising:
a housing provided with a medicament inlet;
a movable member channel arranged in the housing;
a movable member arranged in the movable member channel, the movable member channel having a fluid inlet in fluid communication with the medicament inlet and a fluid outlet, wherein the movable member is configured to move in the movable member channel, by a built-up overpressure inside the movable member channel relative to a pressure on the movable member outside the movable member channel, from a first movable member position in which the movable member blocks the fluid outlet to a second movable member position in which the fluid outlet is in fluid connection with the fluid inlet;
a skin penetration device configured to move from a starting position to a skin penetration position;
a conduit connected to the skin penetration device and to the fluid outlet; and
a movable structure pretensioned in a direction away from the movable member,
wherein the movable member is configured to engage with the structure when the movable member is in the first movable member position, preventing the structure from moving away from the movable member, and
wherein the movable member is configured to disengage from the structure when the movable member is moved towards the second movable member position, causing the pretensioned structure to move away from the movable member to an intermediate position, bringing the skin penetration device from the starting position to the skin penetration position.

16. The on-body injection device of claim 15, comprising a gas filter configured to allow air to pass through and to block liquid from passing through, wherein the movable member has an internal channel extending from a lower end of the movable member along a longitudinal axis of the movable member, and wherein the internal channel is in fluid communication with the fluid inlet, and wherein the gas filter seals the internal channel.

17. The on-body injection device of claim 15, wherein the structure is configured to move away from the movable member in a direction that is perpendicular to the movement of the movable member from the first movable member position to the second movable member position.

18. The on-body injection device of claim 15, comprising a sealing ring arranged around the movable member, the sealing ring being configured to provide fluid sealing between the movable member channel and the movable member.

19. The on-body injection device of claim 15, wherein one of the movable member and the structure comprises a recess and the other one of the movable member and the structure has a heel configured to engage with the recess to engage the movable member and the structure in the first movable member position of the movable member.

20. The on-body injection device of claim 15, wherein the housing has a body side wall configured to bear against the skin of a user, and a distal side wall opposite to the body side wall, wherein the movable member is configured to move in a direction from the body side wall towards the distal side wall when the movable member is moved from the first movable member position to the second movable member position.

21. The on-body injection device of claim 20, wherein the body side wall is provided with a through-opening, wherein the skin penetration device comprises a cannula configured to move out from the housing via the through-opening when the skin penetration device is moved from the starting position to the skin penetration position.

22. The on-body injection device of claim 20, wherein the housing has a sidewall connecting the body side wall and the distal side wall, wherein the structure is configured to move towards the sidewall.

23. The on-body injection device of claim 15, wherein the structure has a first cam surface configured to cooperate with the skin penetration device to cause the skin penetration device to move from the starting position to the skin penetration position as the structure is moved to the intermediate position.

24. The on-body injection device of claim 15, wherein the structure is by pretension configured to continue to move from the intermediate position to a final structure position, causing the structure to bring part of the skin penetration device from the skin penetration position to a final position.

25. The on-body injection device of claim 24, wherein the final position is the same as the starting position.

26. The on-body injection device of claim 24, wherein the structure has a second cam surface configured to cooperate with the skin penetration device to cause the skin penetration device to move from the skin penetration position to the final position.

27. A medicament delivery device comprising the on-body injection device lf claim 15.

28. The medicament delivery device of claim 27, comprising:
a hose connected to the medicament inlet of the on-body injection device; and
a medicament pump device comprising:
a medicament pump device housing; and
a motor and a pump arranged in the medicament pump device housing,
wherein the motor is configured to drive the pump, and wherein the pump is configured to pump medicament to the on-body injection device via the hose.
